(19) United States
(12) Patent Application Publication
(10) Pub. No.: US 2024/0038336 A1
(43) Pub. Date: Feb. 1, 2024

(54) PREDICTING CELL FREE DNA SHEDDING

(71) Applicant: INTERNATIONAL BUSINESS MACHINES CORPORATION, ARMONK, NY (US)

(72) Inventors: Kahn Rhrissorrakrai, Woodside, NY (US); FILIPPO UTRO, Pleasantville, NY (US); Chaya Levovitz, New York, NY (US); Laxmi Parida, Mohegan Lake, NY (US)

(21) Appl. No.: 17/814,850

(22) Filed: Jul. 26, 2022

(51) Int. Cl. G16B 35/10; G16H 50/20

(52) U.S. Cl. CPC G16B 35/10; G16H 50/20

(57) ABSTRACT

A method is provided for training a predicting cfDNA shedding model using a plurality of lesion and cfDNA datasets. A new cfDNA shedding sample and the plurality of lesion and cfDNA datasets are clustered to predict a shedding pattern. A diagnostic type is determined for a subsequent cfDNA shedding sample based on the predicted shedding pattern.

### BACKGROUND

Exemplary embodiments of the present inventive concept relate to cell free DNA shedding (cfDNA), and more particularly, to predicting cfDNA shedding.

Cancer patients require regular monitoring to evaluate their clinical treatment response. While proximal protein levels and standard blood tests can be used for treatment response monitoring, tracking of a tumour (lesion) molecular profile via biopsy provides much greater resolution regarding the tumour and emergent mechanisms of resistance. However, tissue biopsies are highly invasive and unideal for routine treatment response monitoring, particularly in the case of a patient exhibiting multiple lesions and/or inaccessible lesions (e.g., brain lesions). Thus, liquid biopsies (e.g., urine, stool, blood, etc.) are becoming the preferred method for lesion molecular monitoring. Although methods exist to monitor the contributions of different lesions into the cfDNA, lesions may shed differently into cfDNA depending on the time, type, location, and molecular profile. Thus, direct tissue biopsies may still be required to obtain the most accurate understanding of a patient's clinical treatment response. However, at present, there is no way to predict when a liquid biopsy versus a tissue biopsy for a lesion should be performed.

### SUMMARY

Exemplary embodiments of the present inventive concept relate to a method, a computer program product, and a system for predicting cfDNA shedding.

According to an exemplary embodiment of the present inventive concept, A method is provided for training a predicting cfDNA shedding model using a plurality of lesion and cfDNA datasets. A new cfDNA shedding sample and the plurality of lesion and cfDNA datasets are clustered to predict a shedding pattern. A diagnostic type is determined for a subsequent cfDNA shedding sample based on the predicted shedding pattern.

According to an exemplary embodiment of the present inventive concept, a computer program product is provided for predicting cfDNA shedding. The computer program product includes one or more non-transitory computer-readable storage media and program instructions stored on the one or more non-transitory computer-readable storage media capable of performing a method. The method is provided for training a predicting cfDNA shedding model using a plurality of lesion and cfDNA datasets. A new cfDNA shedding sample and the plurality of lesion and cfDNA datasets are clustered to predict a shedding pattern. A diagnostic type is determined for a subsequent cfDNA shedding sample based on the predicted shedding pattern.

According to an exemplary embodiment of the present inventive concept, a computer system is provided for predicting cfDNA shedding. The system includes one or more computer processors, one or more computer-readable storage media, and program instructions stored on the one or more of the computer-readable storage media for execution by at least one of the one or more processors capable of performing a method. The method is provided for training a predicting cfDNA shedding model using a plurality of lesion and cfDNA datasets. A new cfDNA shedding sample and the plurality of lesion and cfDNA datasets are clustered to predict a shedding pattern. A diagnostic type is determined for a subsequent cfDNA shedding sample based on the predicted shedding pattern.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of a predicting cfDNA shedding system 100, in accordance with an exemplary embodiment of the present inventive concept.

FIG. 2 illustrates a flowchart of predicting cfDNA shedding 200, in accordance with an exemplary embodiment of the present inventive concept.

FIG. 2A illustrates a diagram of predicting cfDNA shedding 200 according to an exemplary embodiment of the present inventive concept.

FIG. 3 illustrates a block diagram depicting the hardware components included in the predicting cfDNA shedding system 100 of FIG. 1, in accordance with an exemplary embodiment of the present inventive concept.

FIG. 4 illustrates a cloud computing environment, in accordance with an exemplary embodiment of the present inventive concept.

FIG. 5 illustrates abstraction model layers, in accordance with an exemplary embodiment of the present inventive concept.

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION OF THE DRAWINGS

Exemplary embodiments of the present inventive concept are disclosed hereafter. However, it shall be understood that the scope of the present inventive concept is dictated by the claims. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

As aforementioned, there is presently no way to accurately predict cfDNA shedding of a lesion. Consequently, there is also no reliable means to determine whether a minimally invasive liquid biopsy will suffice, or a more invasive tissue biopsy is required. The presence of multiple lesions in a patient can exacerbate these problems. An advantage of the present invention is that it enables users to reliably predict cfDNA shedding for lesions and patient prognosis trajectory. Consequently, highly invasive tissue biopsies and concomitant patient discomfort can be minimized without compromising lesion detection accuracy.

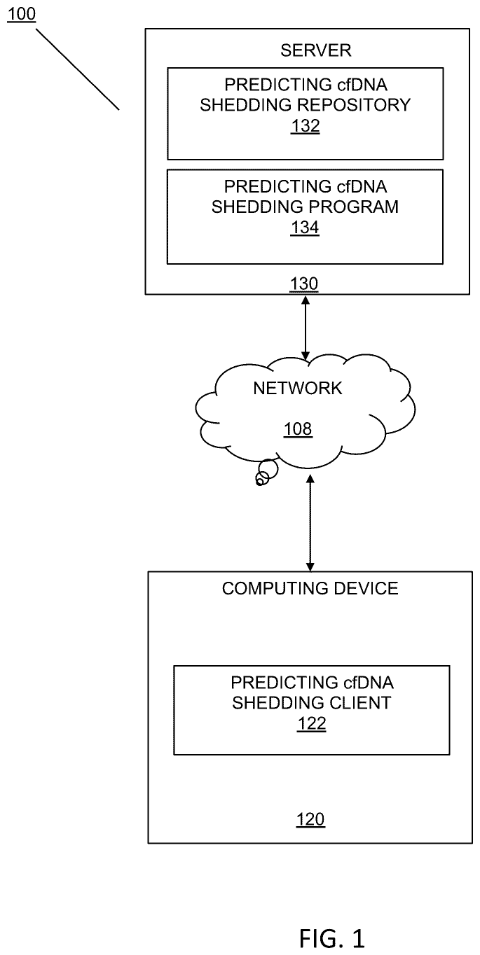

FIG. 1 depicts a predicting cfDNA shedding system 100, in accordance with an exemplary embodiment of the present inventive concept.

The predicting cfDNA shedding system 100 may include a computing device 120 and a predicting cfDNA shedding server 130, which may all be interconnected via a network 108. Programming and data content may be stored and accessed remotely across several servers via the network 108. Alternatively, programming and data may be stored locally on as few as one physical computing device 120 or stored amongst multiple computing devices.

According to the exemplary embodiment of the present inventive concept depicted in FIG. 1, the network 108 may be a communication channel capable of transferring data between connected devices. The network 108 may be the Internet, representing a worldwide collection of networks 108 and gateways to support communications between devices connected to the Internet. Moreover, the network 108 may utilize various types of connections such as wired, wireless, fiber optic, etc., which may be implemented as an intranet network, a local area network (LAN), a wide area network (WAN), or a combination thereof. The network 108 may be a Bluetooth network, a Wi-Fi network, or a combination thereof. The network 108 may operate in frequencies including 2.4 GHz and 5 GHz internet, near-field communication, Z-Wave, Zigbee, etc. The network 108 may be a telecommunications network used to facilitate telephone calls between two or more parties comprising a landline network, a wireless network, a closed network, a satellite network, or a combination thereof. In general, the network 108 may represent any combination of connections and protocols that will support communications between connected devices.

The computing device 120 may include a predicting cfDNA shedding client 122, and may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. The computing device 120 may be connected to a microphone, a camera, and/or various other equipment used in lesion detection/treatment (e.g., apparatuses for liquid biopsy performance/analysis, tissue biopsy performance/analysis, imaging, etc.). Although the computing device 120 is shown as a single device, the computing device 120 may be comprised of a cluster or plurality of computing devices, in a modular manner, etc., working together or working independently.

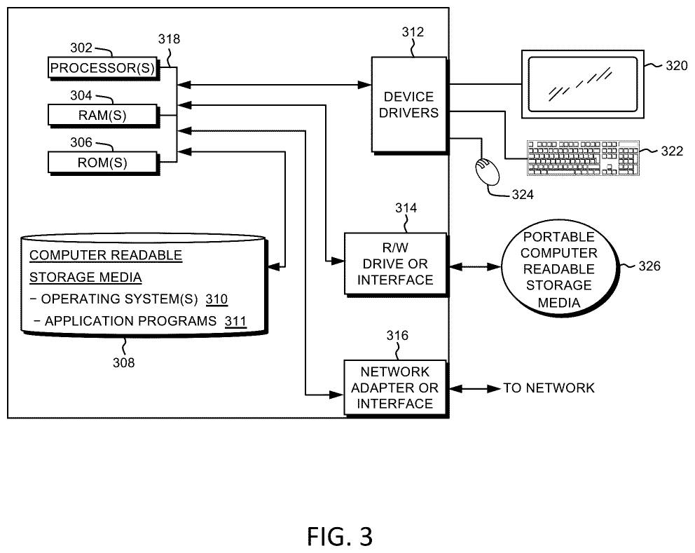

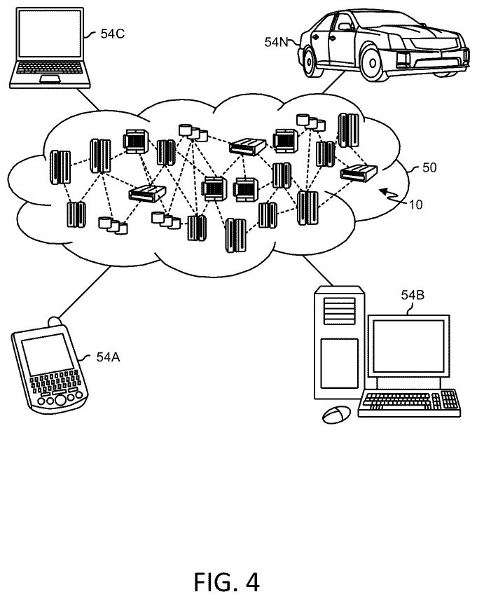

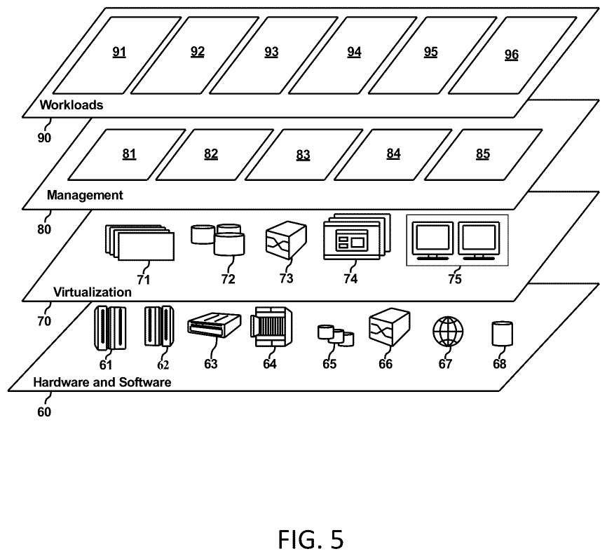

The computing device 120 is described in greater detail as a hardware implementation with reference to FIG. 3, as part of a cloud implementation with reference to FIG. 4, and/or as utilizing functional abstraction layers for processing with reference to FIG. 5.

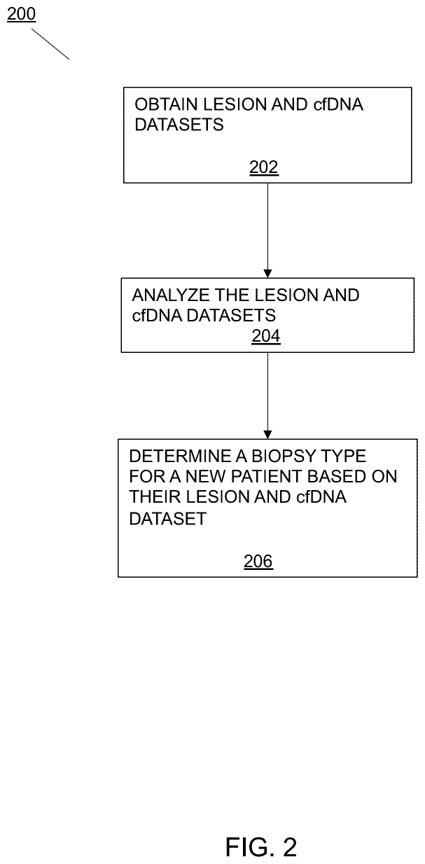

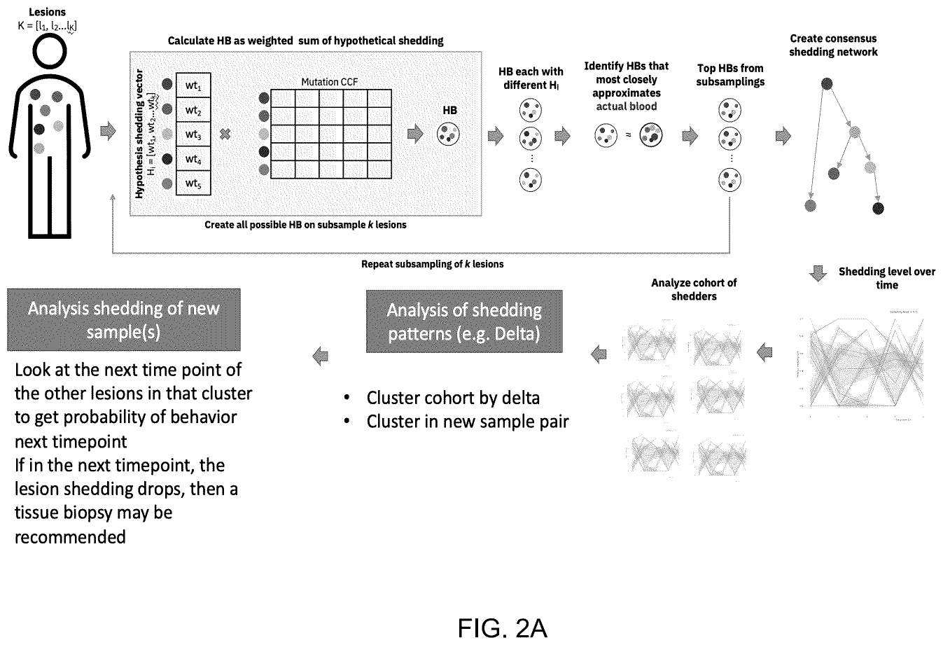

The predicting cfDNA shedding client 122 may act as a client in a client-server relationship with a server, for example the predicting cfDNA shedding server 130. The predicting cfDNA shedding client 122 may be a software and/or a hardware application capable of communicating with and providing a user interface for a user to interact with the predicting cfDNA shedding server 130 and/or other computing devices via the network 108. Moreover, the predicting cfDNA shedding client 122 may be capable of transferring data between the computing device 120 and other computer devices/servers via the network 108. The predicting cfDNA shedding client 122 may utilize various wired and wireless connection protocols for data transmission and exchange, including Bluetooth, 2.4 GHz and 5 GHz internet, near-field communication, etc. The predicting cfDNA shedding client 122 is described in greater detail with respect to FIGS. 2-5.

The predicting cfDNA shedding server 130 may include a predicting cfDNA shedding repository 132 for storing various data (e.g., cfDNA sample compositions and measurements, hypothesis blood (HB) samples/matches to cfDNA, consensus shedding networks, shedding level over time graphs, analyzed cohort of shedders, a predicting cfDNA shedding model, etc.) and a predicting cfDNA shedding program 134. The predicting cfDNA shedding program 134 may obtain lesion and cfDNA datasets; analyze the lesion and cfDNA datasets; and determine a biopsy type for a new patient based on their lesion and cfDNA dataset. The predicting cfDNA shedding server 130 may act as a server in a client-server relationship with a client (e.g., the predicting cfDNA shedding client 122). The predicting cfDNA shedding server 130 may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an IoT device, or any other electronic device or computing system capable of sending and receiving data to and from other computing devices. Although the predicting cfDNA shedding server 130 is shown as a single computing device, the present inventive concept is not limited thereto. For example, the predicting cfDNA shedding server 130 may be comprised of a cluster or plurality of computing devices, in a modular manner, etc., working together or working independently.

The predicting cfDNA shedding server 130 is described in greater detail as a hardware implementation with reference to FIG. 3, as part of a cloud implementation with reference to FIG. 4, and/or as utilizing functional abstraction layers for processing with reference to FIG. 5. The predicting cfDNA shedding program 134 and/or the predicting cfDNA shedding client 122 may be software and/or hardware programs that may predict cfDNA shedding as discussed in further detail with reference to FIGS. 2-5.

FIG. 2 illustrates the flowchart of predicting cfDNA shedding 200, in accordance with an exemplary embodiment of the present inventive concept.

The predicting cfDNA shedding program 134 may obtain lesion and cfDNA datasets (step 202). The predicting cfDNA shedding program 134 may obtain a plurality of lesion and cfDNA datasets corresponding to a plurality of patients. Each lesion and cfDNA dataset may include a plurality of cfDNA sample (e.g., liquid biopsy) measurements and/or tissue biopsy measurements corresponding to at least one lesion. The lesion and cfDNA dataset may include at least two cfDNA sample measurements taken at different times (e.g., treatment monitoring intervals) over a predetermined period (e.g., at least a portion of an individual patient's treatment course for the at least one lesion). The lesion and cfDNA dataset may further include patient lesion profiles (e.g., quantity, type, location, stage, onset, etc.) and/or other relevant patient clinical characteristics (e.g., family history, personal health history, prior remission status, etc.). The predicting cfDNA shedding program 134 may obtain the lesion and cfDNA datasets via user upload/download and/or transmission of anonymous patient data from computing devices 120 used in a clinical setting via the network 108. The predicting cfDNA shedding program 134 may anonymize (e.g., substitute or remove personal identifying information) and/or extract relevant features (e.g., lesion profiles, patient clinical characteristics, cfDNA sample measurements, imaging/results, tissue biopsy measurements/results, etc.) from the obtained lesion and cfDNA datasets using machine learning (e.g., natural language processing (NLP), optical character recognition (OCR), etc.). A predicting cfDNA shedding model may be trained using the lesion and cfDNA datasets. The lesion and cfDNA datasets may be stored in the predicting cfDNA shedding repository 132.

For example, an oncologist has the predicting cfDNA shedding client 122 installed on a clinic-based computing device 120. The oncologist uploads lesion and cfDNA datasets to the predicting cfDNA shedding program 134 for 25 patients treating at the clinic. The predicting cfDNA shedding program 134 erase any remnants of personally identifying information from the lesion and cfDNA datasets. Analysis reveals that 5 of the patients suffer from lesions located in each of a lung, the stomach, and the epidermis, and have only presented for initial consultations thus far.

The predicting cfDNA shedding program 134 may analyze the lesion and cfDNA datasets (step 204). The predicting cfDNA shedding program 134 may use a lesion shedding model (LSM) framework. HB samples may be generated. The HB samples may be used for matching with collected cfDNA samples. Determining the HB sample match to cfDNA sample measurements may enable calculation of relative lesion contribution (e.g., weights, mutations, representative molecules, etc.) and/or shedding related to the overall cfDNA sample and the generation of a consensus shedding network. The consensus shedding network may represent a robust representation of patient lesion shedding derived from stochastic sampling of lesion subsets which the predicting cfDNA shedding program 134 may analyze. This sampling may be performed numerous times, and consensus shedding networks may be generated from the frequencies of the partial orderings of the lesions.

The predicting cfDNA shedding program 134 may generate a “shedding level over time” (SLOT) graph which illustrates a shedding pattern for each lesion and cfDNA dataset based on the consensus shedding network and corresponding empirical data from the lesion and cfDNA dataset. A user (e.g., physician) may input clinically significant annotations related to cfDNA sample measurements (e.g., deviations/alignments with expected clinical treatment course, confounding measurement factors, lesion location and/or measurements, liquid biopsy versus tissue biopsy, undetectable cfDNA shedding which still warranted a subsequent tissue biopsy, detectable cfDNA shedding which became undetectable at a subsequent biopsy, remission, progression, etc.) to train the predicting cfDNA shedding model. The SLOT graph may be visually displayed to the user with annotations. The annotations may include demarcating lesion data points, locations, line graphs (e.g., color-coding, data point symbols, etc.) and/or displaying the learned clinically significant annotations.

In an embodiment, the predicting cfDNA program 134 may analyze differences between similar patients with different lesion profiles and/or cfDNA/HB samples to glean insights. For example, the insights may include calculating the weighted effect of patient clinical characteristics (or lack thereof), confounding measurement factors, variations in lesion profile and/or lesion attributes (e.g., size, age, precise location), etc.

The lesion and cfDNA datasets, relevant patient clinical characteristics, HB samples, gleaned insights, consensus shedding network, SLOT graphs, annotations, etc. may be used for training the predicting cfDNA shedding model and may be stored in the predicting cfDNA shedding repository 132.

For example, the 5 patients each suffering from lung, stomach, and epidermis lesions continue to present to the oncologist for the duration of their treatment course and their respective lesion and cfDNA datasets are updated accordingly. At the second visit of the patients' treatment courses, their respective lung lesions exhibit very low levels of shedding/unexpected dynamics, prompting tissue biopsies and imaging. The tissue biopsies and imaging demonstrate that the patients' respective lung lesions have not resolved, which is also corroborated by elevated lung lesion attributed shedding rates in respective cfDNA samples from liquid biopsies taken at a third treatment monitoring visit. The oncologist annotates that in each case, the lung lesion is not merely in the thoracic region generally, but the right lung precisely. Furthermore, the oncologist annotates that the right lung lesion shedding resumption coincides with abrupt lesion growth based on observations at the subsequent visit. The oncologist inputs these clinically significant annotation updates, and the predicting cfDNA shedding program 134 analyzes them with the lesion and cfDNA datasets and learns accordingly. The predicting cfDNA shedding program 134 may generate HB sample matches and comprehensive SLOT graphs for the 5 patients.

The predicting cfDNA shedding program 134 may determine a biopsy type for a new patient based on their lesion and cfDNA dataset (step 206). The new patient may have at least one cfDNA sample. The at least one new cfDNA sample may be included in a new lesion and cfDNA dataset. The predicting cfDNA shedding program 134 may generate a HB sample(s) and determine a match to the new cfDNA sample(s). At least one of a new cfDNA sample, lesion profile, and/or HB sample may be matched to at least one of a cfDNA sample, lesion profile, and/or HB sample stored in the predicting cfDNA shedding repository 132. Overall patient matches retrieved may be ranked by a similarity score. The similarity score may be based on overall congruence between a new patient's lesion and cfDNA dataset and a prior patient's lesion and cfDNA dataset. Similarity scores below a predetermined threshold may be discarded for the purposes of patient matching. Constituent features (e.g., HB match, lesion profile match, cfDNA sample match) may be prioritized differently in determining a match (e.g., weighted based on user input and/or machine learning analysis gleaned insights). At least one consensus shedding network and SLOT graph may be retrieved and/or generated accordingly. The SLOT graph may display generated annotations relevant to the patient's status and prognosis. The SLOT graph for the new patient may be clustered and analyzed (e.g., via Delta) with the SLOT graph for the at least one stored match (e.g., prior patient, cohort of shedders, etc.). If a discrepancy exists between a stored match's lesion profile and the new patient's lesion profile despite matching cfDNA samples and/or HB samples, a new HB sample, consensus shedding network, and/or SLOT graph may be generated for the new cfDNA sample using at least two measurements. Thus, the consensus shedding network, SLOT, and associated shedding pattern may be determined for the new patient.

In an embodiment, when a new patient has multiple lesions and their lesion profile and/or new cfDNA sample do not sufficiently match a prior patient (e.g., nonidentical lesion types, quantity, sizes, precise locations, cfDNA samples, etc.) the predicting cfDNA shedding program 134 may conflate data from different prior patients into a single hypothetical patient. For example, the predicting cfDNA shedding program 134 may retrieve a nearest, sub-threshold similarity score-based match as a point of departure for generating the hypothetical patient. Alterations to the nearest match may be computed (e.g., adding or subtracting lesions, altering cfDNA relative contribution weights based on size, location, time, etc.). The hypothetical patient may have a scored similarity that is substantially similar to the new patient. The predicting cfDNA shedding program 134 may generate a plurality of hypothetical patients and HB samples exhibiting slight variations in lesion and cfDNA dataset features, SLOT graph, and/or probabilities for cluster analysis. Thus, the hypothetical patient may yield a representative, comprehensive SLOT and a predicted shedding pattern despite an absence of similarity score matches within a predetermined threshold.

Based on the predicted shedding pattern, the predicting cfDNA shedding program 134 may indicate to the user which biopsy type is indicated and/or if imaging is advised at a subsequent treatment monitoring visit. Probabilities may be presented to the user and may indicate the confidence of a biopsy and/or imaging suggestion. Potentially significant differences from at least one cohort member may also be presented to the user. For example, when shedding is below a predetermined threshold, but a subsequent datapoint indicates resumption of shedding, the predicting cfDNA shedding program 134 may suggest a tissue biopsy and/or imaging be performed. When lesion shedding exceeds a predetermined threshold and is projected to remain high at a subsequent datapoint, liquid biopsy may be suggested. When lesion shedding exceeds a predetermined threshold but is projected to plateau or abate at a subsequent datapoint, imaging may be suggested. The predicting cfDNA shedding program 134 may receive user feedback, updates to patient clinical characteristics, and monitor projection accuracy to tune the predicting cfDNA shedding model accordingly. The new patient lesion and cfDNA dataset, analyzed cohort of shedders/SLOT graph/shedding pattern, and the suggested biopsy types may be stored in the predicting cfDNA and lesion repository 132.

For example, a new cancer patient presents to the oncologist at the clinic and their new cfDNA samples are uploaded to the predicting cfDNA shedding program 134. The predicting cfDNA shedding program 134 generates an HB sample and matches the HB sample to the HB samples of the prior 5 patients within a predetermined threshold. The predicting cfDNA shedding program 134 determines that the new patient has lung, stomach, and epidermis lesions, which is corroborated by an independent evaluation by the oncologist. The predicting cfDNA shedding program 134 generates a SLOT graph, clusters it with the existing cohort of shedders (prior 5 patients) and performs Delta analysis. The analyzed cohort of shedders is used by the predicting cfDNA shedding program 134 to then predict the lesion shedding patterns for the new patient. Based on the lesion shedding patterns for the new patient, the predicting cfDNA shedding program 134 suggests that a tissue biopsy, liquid biopsy, and imaging be performed for the lung, epidermis, and stomach lesions, respectively at the next clinical treatment response monitoring appointment.

FIG. 2A illustrates a diagram of predicting cfDNA shedding 200 according to an exemplary embodiment of the present inventive concept.

FIG. 3 illustrates a block diagram depicting the hardware components of the predicting cfDNA shedding system 100 of FIG. 1, in accordance with an exemplary embodiment of the present inventive concept.

It should be appreciated that FIG. 3 provides only an illustration of one implementation and does not imply any limitations regarding the environments in which different embodiments may be implemented. Many modifications to the depicted environment may be made.

Devices used herein may include one or more processors 302, one or more computer-readable RAMs 304, one or more computer-readable ROMs 306, one or more computer readable storage media 308, device drivers 312, read/write drive or interface 314, network adapter or interface 316, all interconnected over a communications fabric 318. Communications fabric 318 may be implemented with any architecture designed for passing data and/or control information between processors (such as microprocessors, communications and network processors, etc.), system memory, peripheral devices, and any other hardware components within a system.

One or more operating systems 310, and one or more application programs 311 are stored on one or more of the computer readable storage media 308 for execution by one or more of the processors 302 via one or more of the respective RAMs 304 (which typically include cache memory). In the illustrated embodiment, each of the computer readable storage media 308 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a R/W drive or interface 314 to read from and write to one or more portable computer readable storage media 326. Application programs 311 on said devices may be stored on one or more of the portable computer readable storage media 326, read via the respective R/W drive or interface 314 and loaded into the respective computer readable storage media 308.

Devices used herein may also include a network adapter or interface 316, such as a TCP/IP adapter card or wireless communication adapter (such as a 4G wireless communication adapter using OFDMA technology). Application programs 311 on said computing devices may be downloaded to the computing device from an external computer or external storage device via a network (for example, the Internet, a local area network or other wide area network or wireless network) and network adapter or interface 316. From the network adapter or interface 316, the programs may be loaded onto computer readable storage media 308. The network may comprise copper wires, optical fibers, wireless transmission, routers, firewalls, switches, gateway computers and/or edge servers.

Devices used herein may also include a display screen 320, a keyboard or keypad 322, and a computer mouse or touchpad 324. Device drivers 312 interface to display screen 320 for imaging, to keyboard or keypad 322, to computer mouse or touchpad 324, and/or to display screen 320 for pressure sensing of alphanumeric character entry and user selections. The device drivers 312, R/W drive or interface 314 and network adapter or interface 316 may comprise hardware and software (stored on computer readable storage media 308 and/or ROM 306).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

It is to be understood that although this disclosure includes a detailed description on cloud computing, implementation of the teachings recited herein are not limited to a cloud computing environment. Rather, the exemplary embodiments of the present inventive concept are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 4 illustrates a cloud computing environment, in accordance with an exemplary embodiment of the present inventive concept.

As shown, cloud computing environment 50 may include one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

FIG. 5 illustrates abstraction model layers, in accordance with an exemplary embodiment of the present inventive concept.

Referring now to FIG. 5, a set of functional abstraction layers provided by cloud computing environment 50 (FIG. 4) is shown. It should be understood in advance that the components, layers, and functions shown in FIG. 5 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfilment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and predicting cfDNA shedding 96.

The exemplary embodiments of the present inventive concept may be a system, a method, and/or a computer program product at any possible technical detail level of integration. The computer program product may include a computer readable storage medium (or media) having computer readable program instructions thereon for causing a processor to carry out aspects of the present inventive concept.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present inventive concept may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present inventive concept.

Aspects of the present inventive concept are described herein with reference to flowchart illustrations and/or block diagrams of methods, apparatus (systems), and computer program products according to exemplary embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present inventive concept. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

1. A method for predicting cell free DNA (cfDNA) shedding, the method comprising:
training a predicting cfDNA shedding model using a plurality of lesion and cfDNA datasets;
clustering a new cfDNA shedding sample and the plurality of lesion and cfDNA datasets to predict a shedding pattern; and
determining a diagnostic type for a subsequent cfDNA shedding sample based on the predicted shedding pattern.

2. The method of claim 1, wherein the training of the predicting cfDNA shedding model using the plurality of lesion and cfDNA datasets includes performing lesion shedding analysis (LSM).

3. The method of claim 1, wherein the determined diagnostic type is at least one of a urine sample, stool sample, a blood sample, radiographic imaging, and a tissue biopsy.

4. The method of claim 1, wherein the determined diagnostic type is selected according to a predetermined threshold of predicted shedding for the subsequent cfDNA shedding sample.

5. The method of claim 1,
wherein the new cfDNA shedding sample is associated with a plurality of lesions, and
wherein each lesion of the plurality of lesions has a different determined diagnostic type for the subsequent cfDNA shedding sample based on a corresponding shedding pattern.

6. The method of claim 5, further comprising:
determining relative cfDNA contributions for each lesion of the plurality of lesions into the new cfDNA shedding sample,
wherein the relative cfDNA contributions depend on at least one of time, location, lesion type, and molecular profile.

7. The method of claim 2, further comprising:
generating a hypothesis blood that matches each of the lesion and cfDNA datasets;
generating a consensus shedding network and a shedding level over time from the hypothesis blood;
determining a cohort of shedders from the shedding level of time; and
clustering the new cfDNA shedding sample with a cohort of substantially similar shedders over time to predict the shedding pattern.

8. A computer program product for predicting cfDNA shedding, the computer program product comprising:
one or more non-transitory computer-readable storage media and program instructions stored on the one or more non-transitory computer-readable storage media capable of performing a method, the method comprising:
training a predicting cfDNA shedding model using a plurality of lesion and cfDNA datasets;
clustering a new cfDNA shedding sample and the plurality of lesion and cfDNA datasets to predict a shedding pattern; and
determining a diagnostic type for a subsequent cfDNA shedding sample based on the predicted shedding pattern.

9. The computer program product of claim 8, wherein the training of the predicting cfDNA shedding model using the plurality of lesion and cfDNA datasets includes performing lesion shedding analysis (LSM).

10. The computer program product of claim 8, wherein the determined diagnostic type is at least one of a urine sample, stool sample, a blood sample, radiographic imaging, and a tissue biopsy.

11. The computer program product of claim 8, wherein the determined diagnostic type is selected according to a predetermined threshold of predicted shedding for the subsequent cfDNA shedding sample.

12. The computer program product of claim 8,
wherein the new cfDNA shedding sample is associated with a plurality of lesions, and
wherein each lesion of the plurality of lesions has a different determined diagnostic type for the subsequent cfDNA shedding sample based on a corresponding shedding pattern.

13. The computer program product of claim 12, further comprising:
determining relative cfDNA contributions for each lesion of the plurality of lesions into the new cfDNA shedding sample,
wherein the relative cfDNA contributions depend on at least one of time, location, lesion type, and molecular profile.

14. The computer program product of claim 9, further comprising:
generating a hypothesis blood that matches each of the lesion and cfDNA datasets;
generating a consensus shedding network and a shedding level over time from the hypothesis blood;
determining a cohort of shedders from the shedding level of time; and
clustering the new cfDNA shedding sample with a cohort of substantially similar shedders over time to predict the shedding pattern.

15. A computer system for predicting cfDNA shedding, the system comprising:
one or more computer processors, one or more computer-readable storage media, and program instructions stored on the one or more of the computer-readable storage media for execution by at least one of the one or more processors capable of performing a method, the method comprising:
training a predicting cfDNA shedding model using a plurality of lesion and cfDNA datasets;
clustering a new cfDNA shedding sample and the plurality of lesion and cfDNA datasets to predict a shedding pattern; and
determining a diagnostic type for a subsequent cfDNA shedding sample based on the predicted shedding pattern.

16. The computer system of claim 15, wherein the training of the predicting cfDNA shedding model using the plurality of lesion and cfDNA datasets includes performing lesion shedding analysis (LSM).

17. The computer system of claim 15, wherein the determined diagnostic type is at least one of a urine sample, stool sample, a blood sample, radiographic imaging, and a tissue biopsy.

18. The computer system of claim 15, wherein the determined diagnostic type is selected according to a predetermined threshold of predicted shedding for the subsequent cfDNA shedding sample.

19. The computer system of claim 15,
wherein the new cfDNA shedding sample is associated with a plurality of lesions, and
wherein each lesion of the plurality of lesions has a different determined diagnostic type for the subsequent cfDNA shedding sample based on a corresponding shedding pattern.

20. The computer system of claim 19, further comprising:
determining relative cfDNA contributions for each lesion of the plurality of lesions into the new cfDNA shedding sample,
wherein the relative cfDNA contributions depend on at least one of time, location, lesion type, and molecular profile.
